(19) United States
(12) Patent Application Publication
(10) Pub. No.: US 2023/0174575 A1
(43) Pub. Date: Jun. 8, 2023

(54) A NEW PROCESS OF PURIFICATION OF PROTEINS FROM HEN EGG WHITE AND THE USE OF THE SAME AS ANTIVIRAL AGENT AGAINST SARS-COV-2

(71) Applicant: BIOSEUTICA B.V., Zeewolde, BD (NL)

(72) Inventors: Valerio Maria Ferrari, Milano (MI) (IT); Maria Carla Baggio, Saronno (VA) (IT); Paride Grisenti, Milano (IT)

(73) Assignee: BIOSEUTICA B.V., Zeewolde, BD (NL)

(21) Appl. No.: 17/998,363

(22) PCT Filed: May 11, 2021
(86) PCT No.: PCT/IB2021/053999

§ 371 Date: Nov. 10, 2022

Related U.S. Application Data
(60) Provisional application No. 63/022,638, filed on May 11, 2020.

(30) Foreign Application Priority Data
Jul. 9, 2020 (IT) 102020000016663

(51) Int. Cl. C07K 1/36; A61K 38/47; A61K 38/17; A61P 31/14

(52) U.S. Cl. CPC C07K 1/36; A61K 38/47; A61K 38/1703; A61P 31/14

(57) ABSTRACT

The present invention describes a novel process for the production of lysozyme HCl from hen egg white (HEW) with high chemical purity, and the use of said antiviral agent against SARS-CoV-2, optionally combined with other antivirals and/or immunosuppressants and ovotransferrin.

### FIELD OF INVENTION

The present invention discloses a novel process for the production of lysozyme HCl from hen egg white (HEW) with high chemical purity, and the use of said antiviral agent against SARS-CoV-2, optionally combined with other antivirals and/or immunosuppressants and ovotransferrin.

### TECHNICAL BACKGROUND

The protein composition of HEW has not been fully elucidated. The mixture of proteins presents in HEW is particularly complex, and presents some particular analytical problems because they have very different molecular weights (ranging from 12.7×10³ to 240×10⁶ daltons); their concentrations in egg white are also very different, ovalbumin being the most abundant protein. Proteomic techniques have been applied to HEW analysis to identify and quantify said protein composition. In addition to ovalbumin, which accounts for about 50% of the total proteins present in HEW, other proteins are represented by ovotransferrin, ovomucoid, avidin, lysozyme and ovoglobulin. Specifically, lysozyme and ovotransferrin represent 3.5 and 12-13% respectively of the total proteins present in HEW (J. Agric. Food Chem, 2001, 49, 4553-456).

Lysozyme (also known as muramidase) is a mucolytic enzyme with antibiotic and antiviral properties, first discovered by Alexander Fleming (Proc. Roy. Soc. London 93B, 306 (1922)). Lysozyme is also widespread in nature, being found not only in HEW but also in tears, nasal mucus, milk, saliva, blood serum, numerous tissues ancovid secretions of various animals, both vertebrates and invertebrates, in some moulds, and in the latex of various plants. Because of their different origins, different types of lysozymes have been identified which have the common characteristic of cleaving the glycoside β-(1,4) bonds between N-acetylmuramic acid and N-acetyl glucosamine in peptidoglycan, the main polymer of the bacterial cell wall. Said hydrolytic enzymes belong to the glycosylase family, and are identified by the Enzyme Commission (EC) with the number 3.2.1.17. HEW lysozyme has a molecular weight of about 14,836 daltons, and its primary, secondary and tertiary structure was fully clarified in 1963 (Canfield, R. E. et al. Journal of Biological Chemistry, 240 (5), 997-2002; Blake CCF et al. Nature, 196, 1173, 1962).

Ovotransferrin (also known as conalbumin) is a protein present in HEW, which was described for the first time in 1900 (Osborne, Campbell, J. Am. Chem. Soc. 22, 422 (1900); its purification by other proteins present in egg white was described for the first time in 1940 (Longworth et al., Ibid. 62, 2580 (1940)). The primary structure of hen ovotransferrin, and its purification, characterisation and iron ion binding characteristic, have been known since 1982 (J. Williams et al., Eur. J. Biochem. 122, 297 (1982); W.-M. Keung et al., J. Biol. Chem. 257, 1177, 1184 (1982)). Said protein has a molecular weight of about 76,000 daltons, and possesses antibacterial activity (P. Valenti et al., Antimicrob. Ag. Chemother. 21, 840 (1982)) and antiviral activity (F. Giansanti et al. Biochem. Biophys. Ris. Comm. 331, (2005), 69-73). Moreover, its characteristic of binding/releasing iron is undergoing evaluation with a view to its use as an iron supplement for humans (F. Giansanti et al. Biochimica and Biophysica Acta. 2011, 1820 (3), 218-25).

The use of lysozyme as an active ingredient requires the study and development on an industrial scale of purification methods that offer products with high chromatographic purity and, in view of its natural origin, potentially free of avian viruses (such as Avian avulavirus 1, and influenza H5N1, H7N1 and H7N9). Various examples of laboratory procedures for isolation of lysozyme from HEW by precipitation with salts or solvents have already been described, involving some drawbacks due to denaturation of the proteins or low purity (Linz R. et al. Comptes Rendus des Seances de la Societe de Biologie et de Ses Filiales, 26, 1279-80; Alderton et al., J. Biol. Chem. 157, 43 (1945); Alderton, Fevold, ibid. 164, 1 (1946); Biochem. Prepns 1, 67 (1949); Sophianopoulos et al., J. Biol. Chem. 237, 1107 (1962)).

The purification process of HEW proteins using liquid chromatography, and in particular ion-exchange chromatography, has proved more effective, because in this case, separation depends on the charge of the proteins and the ionic strength of the mobile phase. However, this approach involves some drawbacks for large-scale production, operating with undiluted HEW, because the density of said product creates adsorption problems and causes a low elution flow rate; moreover, the data reported in the literature confirm that in order to obtain acceptable chromatographic separation among egg proteins, it is necessary to operate below the capacity of the resin, which involves the need to work with large amounts of stationary phases. For example, using an ion-exchange column (with a quaternary ammonium resin such as Q Sepharose® Fast Flow), with gradient elution (TrisHCl buffer at pH 9 and 0.3% aqueous solution of NaCl), the recovery of lysozyme is very low (60%). This approach, developed to allow separation between lysozyme and ovotransferrin, offers acceptable chromatographic purity for lysozyme (99 and 88%, divided into two peaks), whereas the chromatographic purity of the ovotransferrin isolated is only 75% (Vachier, M C et al. Journal of Chromatography B, 664 (1995), 201-210). Moreover, the selection of a different ion-exchange resin, in other words an IRC50 resin (a weakly acidic resin with carboxylic acid functionality), and a phosphate buffer at pH 7.18 as mobile phase, has given poor results in terms of purity and yields (Stein et al. Ciba Foundation Symposium, Chem. Structure Proteins, 1952, 17-30).

The need for more efficient chromatography processes to prepare HEW lysozyme and ovotransferrin on an industrial scale has led to studies of alternative chromatography approaches, which involve evaluation of affinity chromatography, the use of more efficient resins and, in particular, of cation-exchange resins, the magnetically stabilised fluidised bed, chromatography using surface imprinting techniques and the use of cryogel.

Affinity chromatography was tested in 1993 by Chiang B. H. et al. (Journal of Food Science, 58 (2), 303-6, 1993) and more recently by Federico J. W. et al. (European Food Research and Technology, 231, 181-188 (2010)). This technique uses affinity interaction between lysozyme and N-acetyl-D-glucosamine monomers of chitin. In particular, the process described by Federico J. W. et al. provides a batch purification process for undiluted HEW lysozyme, wherein 80% of the lysozyme is removed from the HEW and the matrix is recovered by filtration through a filter with a total yield of 64%. In said process, a bio-adsorbent composite is used which retains the chitin bonded non-covalently between the layers of a silicon oxide matrix; said matrix was specifically developed by this research group. The commercial unavailability of said stationary phase, the low number of tests carried out to ensure reuse of said stationary phase, and the extremely slow lysozyme absorption kinetics in said stationary phase (about 10 hours) are unsolved problems for possible scaling-up of this method.

Examples of the use of a cation-exchange resin for chromatographic purification of egg white lysozyme have also been reported, but with poor results in terms of purity and yields. For example, in 2003 Hyoung W. K. et al. (Hwahak Konghak, 41 (3), 332-336, 2003) evaluated the performance of a weak cation-exchange resin with different elution with a NaCl gradient which provides low yields and purity, as demonstrated by the SDS-PAGE analyses reported in the same document. Even the use of a strong cation-exchange resin, such as SP Sepharose FF, did not improve the performance of chromatographic purification of lysozyme; the total lysozyme recovery yields amounted to 80% in a process simulation starting with diluted HEW wherein ovomucin had previously been separated by precipitation, and the filtrate then purified with said SP Sepharose FF resin (Biotechnology Progress, 27 (3), 733-43, 2011).

More recently, purification processes for lysozyme from diluted HEW which use a magnetically stabilized fluidised bed (MSFB) have been reported. For example, using an MSFB with an average spherical particle size of 80-120 μm, prepared by polymerisation in suspension of 2-hydroxyethylmethacrylate in the presence of Fe₂O₄ nanopowder (International Journal of Biological Macromolecules, 41 (3), 234-42, 2007), lysozyme was isolated with 87% purity determined by SDS-PAGE, and 80% recovery. Moreover, exploiting the hydrophobic affinity interaction of hen egg white lysozyme, monosize magnetic microbeads of poly (glycidyl methacrylate-N-methacrylyl-L-tryptophan) (1.6 μm of diameter) were also used. The lysozyme adsorption tests were conducted under different experimental conditions (e.g. lysozyme concentration, temperature and ion resistance) in a magnetically stabilised fluidised bed system (Materials Science & Engineering, C: Materials for Biological Applications, 29 (5), 1627-1634, 2009). However, the need to operate with diluted HEW (because of the small particle size of these stationary phases), the unsolved technical problem of using a magnetic field on large industrial columns, the non-quantitative recovery of lysozyme, and the commercial unavailability of these stationary phases, represent a limitation for scaling-up of these techniques.

The molecular imprinting technique has also been used to separate and purify lysozyme from diluted HEW. Using this technique, poly (glycidyl methacrylate) microbeads were prepared from hydroxyethyl methacrylate to allow the introduction of double polymerisable bonds, by grafting β-cyclodextrin and acrylamide onto the surface as functional monomers (Biomedical Chromatography, 28, (4), 534-540, 2014). Using said polymer with molecular imprinting, the authors found a potential chromatographic enrichment of 80% lysozyme, and postulate a potential industrial use on “real samples”. The size of the stationary phase used (about 5 microns), which requires high operating pressures, and the fact that these tests were performed on diluted HEW, represent unsolved problems for the potential scale-up of this technique.

Finally, one of the latest chromatographic approaches to purification of HEW lysozyme is the use of cryogel. Cryogels are generally networks of supermacroporous gels developed by cryotropic gelation of specific monomers or polymer precursors at sub-zero temperatures (Russ. Chem. Rev., 2002,71, 489-511). This procedure is performed with moderate freezing, storage in the frozen state, and subsequent thawing of colloidal solutions or dispersions containing monomeric or polymeric precursors. The three-dimensional structure of said cryogels is unusual; for example, polyacrylamide cryogels have a spongy morphology, mainly induced by the cryotropic gelation temperature. Various modifications to said cryogel systems for the purification of HEW lysozyme have been developed, which involve coupling a variety of ligands to its surfaces and grafting polymer chains onto the surface of the cryogels; for example, a poly(glycidyl methacrylate-N-methacryloyl-(L)-tryptophan methyl ester) [PGMATryp] bead, produced by polymerisation in dispersion, was loaded onto a poly(2-hydroxyethyl methacrylate) [PHEMA] cryogel to provide a composite cryogel using N,N,N′,N′-tetramethylenediamine and ammonium persulphate at −12° C. (Colloids and Surfaces, B: Biointerfaces, 123, 859-865, (2014)). Said composite cryogel was used as stationary phase for the purification of lysozyme from diluted HEW at pH 7, providing, after elution with a mobile phase at pH 4 containing ethylene glycol, a lysozyme with 85% purity and a 78% yield. The maximum absorption capacity of said composite cryogel was about 350 mg of lysozyme per gram of polymer. Other examples of the use of a composite cryogel incorporated in beads used to purify lysozyme obtained from diluted HEW are a stationary phase of poly(hydroxyethyl methacrylate-N-methacryloyl)-1-phenylalanine with a maximum absorption capacity of 57 mg of lysozyme per gram of polymer, the final recovery yield of which is not reported ((Biotechnology and Applied Biochemistry, 62 (2), 200-7, 2015); and cryogel discs consisting of poly (hydroxyethyl methacrylate), which have a maximum absorption capacity of 103-107 mg of lysozyme per gram of polymers, prepared by immobilising a reactive dye (Cibacron blue F3BA and alkali blue 6B blue) (Applied Biochemistry and Biotechnology, 175 (6), 2795-805, 2015). In this case, the absorption capacity was determined by using said discs in solution and batch treatment, and desorption was performed with an aqueous solution of potassium thiocyanate.

These approaches have the common limitation of using non-commercial stationary phases or cryogel discs and operating on diluted HEW (this is essential for chromatographic purification because of the low particle-size distribution of the stationary phases, namely about 5 microns). Moreover, some of the processes described above use toxic compounds at the desorption stage, such as ethylene glycol or potassium thiocyanate (toxic to humans), which must be thoroughly removed from the end product.

Simpler and more effective purification processes are therefore required to obtain good recovery yields and a lysozyme with a purity suitable for possible use as an active pharmaceutical ingredient (API) in the form of an antiviral and antibacterial agent.

Various publications have studied the potential antiviral and antibacterial activity of lysozyme (human or isolated from HEW), either alone or combined with other antibiotics and antivirals. In particular, the antiviral activity of lysozyme has been studied on parainfluenza virus 3 (NY State Dept. Health, Ann. Rept. Div. Lab. Res., 55, 1961), HIV-1 infection (Appl Biochem Biotechnol (2018) 185: 786-798), Herpes simplex virus type 1 (Current Microbiology, 10 (1984), 35-40), hepatitis A virus (International Journal of Food Microbiology 266 (2018) 104-108), bovine viral diarrhoea virus (Veterinary Research (2019) 15: 318) and poliovirus (https://www.researchgate.net/publishing/320238010).

The mechanism of the antiviral activity of lysozyme on said viral strains is still unclear, but in the case of herpes, when hypolysozymaemia occurs, the infection tends to recur (Fleming's Lysozyme, Edizioni Minerva Medica, 1976), suggesting a direct correlation between the viral infection and the physiological concentration of lysozyme. Although the therapeutic use of lysozyme as an antiviral agent against various viral infections has been tested in vitro and in vivo, no clear evidence has ever been provided for the use of lysozyme isolated from HEW as an API for preventive cytoprotection against a viral infection and/or for the treatment of already infected cells.

In December 2019, the Chinese health authorities reported a group of pneumonia cases of unknown aetiology in the city of Wuhan (Hubei, China), and the agent for said cases was identified as a novel coronavirus (provisionally called 2019-nCoV), against which no effective therapeutic approach has yet been found. The virus responsible for said COVID-19 cases was classified and designated as SARS-CoV-2 by the Coronavirus Study Group (CSG) of the International Committee on Taxonomy of Viruses. More recently, experimental results obtained using Caco-2 cells as model have suggested that “native” lysozyme (purified from human neutrophils and from hen egg white) does not protect against SARS-CoV-2 infection, and in particular that it does not have a direct antiviral activity (Carina Conzelmann et al., An enzyme-based immunodetection assay to quantify SARS-CoV-2 infection, Antiviral Research. Doi: 10.1016/j .antivira1.2020.104882).

It is therefore urgent and necessary to obtain new antiviral agents which are active against SARS-CoV-2, and preferably possess low toxicity and high chemical purity.

### Definitions

Vero cells are a cell line used in cell cultures isolated from renal epithelial cells extracted from an African green monkey (Chlorocebus sp.). Said cell line can replicate through numerous division cycles, and not become senescent.

MOI (Multiplicity of infection) is the ratio between agents (i.e. viruses or bacteria) and infected cells.

ΔCt represents the difference in the cycle threshold (Ct) values of the supernatant of untreated and treated infected cells (ΔCt=Ct of supernatant of untreated cells−Ct of supernatant of infected cells)

PFU (plaque-forming unit) is a measurement used in virology to describe the number of viral particles able to form plaques per unit of volume. The result PFU/mL represents the number of infectious particles in the sample, and is based on the assumption that each plaque formed is representative of an infectious viral particle.

### SUMMARY OF THE INVENTION

The present invention discloses a novel process for the production of lysozyme HCl from HEW with high chemical purity, free of avian viruses like Avian avulavirus 1 and influenza viruses H5N1, H7N1 and H7N9, and the use of the resulting product as antiviral agent against SARS-CoV-2, optionally in combination with other antivirals and immunosuppressants and/or ovotransferrin.

The lysozyme HCl produced according to the invention has proved effective in in vitro tests as a protective agent against SARS-CoV-2 infection, and reduces viral replication in already infected cells.

### Description of the Invention

The invention provides a process for the purification of lysozyme HCl isolated from hen egg white, comprising the following steps:
a) isolation of crude lysozyme base from undiluted hen egg white using a weakly acidic cationic resin under stirring, followed by treatment with an aqueous saline solution;
b) preparation of crude solution of lysozyme HCl;
c) removal of inorganic salts;
d) viral inactivation/antiviral activation
e) isolation of amorphous lysozyme HCl with a spray-drying technique;
f) heat treatment of the lysozyme HCl obtained in step e).

Step a) is preferably conducted with a weakly acidic, polyacrylic, macroporous cationic resin, with a particle size range of 300-1600 μm, preconditioned to pH 7.0-9.0, for example by adding a 15% w/w aqueous solution of sodium carbonate. The relative ratio between undiluted HEW and resin ranges between 8-12 l/l, and the resin is typically maintained under stirring at a stirring speed of up to 60 rpm, and a temperature ranging between 25° C. and 40° C. The weakly acidic cationic resin used in step a) is preferably Purolite® C106EP or equivalent resin having a total capacity ≥2.7 eq/l, preferably treated with a 2-7% NaCl solution at a temperature ranging between 25 and 40° C., preferably 30-35° C.

In step b), the aqueous solution of NaCl eluted in step a) is treated with an aqueous inorganic base until a final pH value ranging between 10 and 11 is reached, at a temperature ranging between 0 and 8° C. for 4-24 hours, to obtain lysozyme base, which is first recovered by filtration, then dissolved in an aqueous solution of hydrochloric acid until a final pH interval of 2.5-3.5 is reached.

The aqueous inorganic base used in step b) is preferably a 4-8% w/v aqueous solution of sodium hydroxide.

In step b), the crude lysozyme base is dispersed in demineralised water at a relative ratio ranging between 1/30 and 1/60 v/v relative to the initial amounts of HEW.

The crude lysozyme hydrochloride solution is corrected in step c) to a final pH interval of 3.0-4.0, using a 2-8% aqueous solution of hydrochloric acid, and ultrafiltered and/or diafiltered to remove the inorganic salts, using ultrafiltration and diafiltration membranes with a cut-off of 10 kdaltons.

The ultrafiltered/diafiltered aqueous solution is then optionally heated in step d) to a temperature ranging between 40° C. and 100° C. for a period of up to 7 days, preferably at 74° C. for 1 hour or at 90° C. for 2-6 minutes.

The solution obtained in step c) or d) is finally heated to 40° C. and treated with a spray-dryer at a desolvation chamber temperature ranging between 160 and 220° C., to give pure amorphous lysozyme hydrochloride.

Step d) can alternatively/simultaneously be conducted on powdered lysozyme hydrochloride obtained after spray-dryer treatment at a temperature ranging between 40° C. and 100° C. for a period of up to 7 days, preferably at 74° C. for 1 hour (step e)).

The invention also provides lysozyme hydrochloride for use as antiviral agent against SARS-CoV-2, for the prevention or treatment of human SARS-CoV-2 infections, optionally combined with other antiviral and/or immunosuppressant agents and/or ovotransferrin. Examples of said agents include chloroquine, favilavir, remdesivir, avigan, tocilizumab, cyclosporin A, sirolimus, everolimus, temsirolimus, mycophenolate mofetil and pimecrolimus.

For the required therapeutic use, the lysozyme hydrochloride is administered orally, topically, by inhalation or injection, intravenously, gastrointestinally, intraperitoneally, intrapleurally, intrabronchially, nasally or rectally using suitable pharmaceutical compositions.

### DETAILED DESCRIPTION OF THE INVENTION

The present invention will now be described in detail, by means of a sample embodiment reported below, on the proviso that variations in the subject-matter, conditions and parameters within the limits defined in the independent claims are comprised in the present invention.

The lysozyme HCl produced according to the invention is isolated from HEW by a purification process involving the following sequence of purification procedures: isolation of crude lysozyme base from other ovoproteins present in HEW, preparation of crude lysozyme HCl solution, removal of inorganic salts, viral inactivation and isolation of solid lysozyme HCl by spray-drying technique, followed by heat treatment.

The lysozyme HCl produced according to said purification process has exhibited antiviral activity against SARS-CoV-2 infection. In particular, lysozyme HCl exhibited a preventive cytoprotective action against SARS-CoV-2 infection on uninfected cells, and antiviral activity on already infected cells.

The crude HEW lysozyme was purified by the following purification process: undiluted HEW was loaded onto a polyacrylic cationic resin with a particle size ranging between 300 and 1600 μm and a capacity of >2.7 eq/l, preconditioned in a pH interval of 9.0-7.0. The relative ratio between HEW and resin ranges between 8 and 12 l/l, and the resin is washed twice with a 1.0-1.5 bed volume of distilled water. The resin was then washed with a 1.5-2.1 bed volume of an aqueous solution of NaCl at a concentration ranging between 2 and 7% and a temperature ranging between 25 and 40° C. When said steps are performed, the resin can optionally be maintained under stirring at a stirring speed of up to 60 rpm.

The fractions eluted with aqueous solution of NaCl were basified at a final pH value ranging between 10 and 11 with an aqueous solution of 4-8% w/v sodium hydroxide, and the resulting mixture was maintained under stirring at 0-8° C. for 4-24 hours. The resulting precipitate was recovered by suction. The wet solid was dispersed under stirring in demineralised water (relative ratio between demineralised water and initial HEW 1/60- 1/100 l/l), and the resulting mixture was maintained under stirring at a temperature ranging between 20 and 50° C. for 30 minutes to 2 hours and corrected to a final pH interval of 2.5-3.5 with a 4-8% w/v aqueous solution of hydrochloric acid. The resulting solution was then heated under stirring at a temperature ranging between 20 and 60° C. for 30 minutes to 2 hours, then cooled, and the final pH value corrected to an interval ranging between 8.0 and 10 by adding a 1-4% w/v aqueous solution of sodium hydroxide. Said solution was treated under stirring for 1-4 hours with activated charcoal (powder) and then filtered (the filter can optionally be washed with demineralised water). The filtrate (and optionally the washing water) was then corrected to a final pH interval of 3.0-4.0 with a 2-8% aqueous solution of hydrochloric acid, and ultrafiltered and/or diafiltered to remove the inorganic salts. The resulting aqueous solution was then optionally heated to a temperature ranging between 40° C. and 100° C. for a period of up to 7 days, then cooled to 40° C., or heated directly to 40° C., and treated with a spray-dryer (desolvation chamber temperature 160-220° C.) to provide pure amorphous lysozyme hydrochloride as an ivory powder (>99% recovery). The above-mentioned step involving heat treatment of lysozyme hydrochloride in solution at a temperature ranging between 40° C. and 100° C. for a period of up to 7 days (preferably at 74° C. for 1 hour) can also alternatively/simultaneously be conducted on powdered lysozyme hydrochloride obtained after spray-drying treatment.

Said controlled heat treatments give the lysozyme prepared by the manufacturing process described herein an antiviral/virucidal activity against SARS-CoV-2 without denaturing the lysozyme which, after said treatments, exhibits unchanged HPLC purity and enzyme activity. In particular, said heat treatments that do not denature the lysozyme are preferably:
1. on solid lysozyme hydrochloride: a treatment at 99° C. for 40 minutes or at 74° C. for 1 hour
2. on lysozyme hydrochloride in aqueous solution: a heat treatment at 90° C. for between 2 and 6 minutes.

The lysozyme HCl obtained by said process is free of avian viruses such as Avian avulavirus 1 and influenza viruses H5N1, H7N1 and H7N9, and exhibits an enzyme assay value on dry matter (enzymatic turbidimetric method)>97.0%, preferably >98.0%, and HPLC purity>99.0%, preferably>99.5%.

Said lysozyme HCl exhibited high antiviral activity against SARS-CoV-2 infection in the in vitro test at a concentration ranging between 0.75 and 1.5 mg/ml.

The activity of lysozyme HCl has been confirmed to be cytoprotective, at practically the same concentrations, on uninfected cells exposed to viral infection and on already infected cells. In fact, at a lysozyme HCl concentration ranging between 0.75 and 1.5 mg/ml, the percentage viral replication was very low, and close to zero.

Moreover, the in vitro antiviral activity of lysozyme HCl combined with ovotransferrin at three different concentrations (1.25, 2.5 and 5 mg/l), discovered by us, proved to be synergic. Under said conditions, the concentration of 1.25 mg/ml of ovotransferrin exhibited the highest synergic effect. The synergic antiviral effect obtained is dose-dependent, and completely eliminated viral replication at the lysozyme concentration of 0.75 mg/ml (at this concentration of lysozyme HCl, and in the absence of ovotransferrin, viral replication was inhibited by about 62%). Ovotransferrin alone was tested at different concentrations, ranging from 10 mg/ml to 1.25 mg/ml, but no antiviral activity was detected in said interval.

The antiviral activity of lysozyme HCl against SARS-CoV-2 is closely correlated with heat treatment of lysozyme HCl (both in solution and in powder form), and exhibited both antiviral and virucidal activity.

### Materials and Methods

The ovotransferrin (product code 501P2001090) used to conduct the in vitro tests is produced by Bioseutica BV (Landbouwweg 83 3899 Zeewolde BD (Netherlands)).

### HPLC Method for Determination of Chromatographic Purity of Lysozyme HCl

HPLC column: TSKgei reverse-phase HPLC column (polymer base, Phenyl-5PW RP), ID 4.6 mm×7.5 cm (10 μm); Tosoh Bioscience
Detector: UV
Wavelength: 281 nm
Preparation of sample: weight 80 mg of lysozyme HCl, diluted to 20 ml with water (4 μg/μl)
Injection volume: 25 μl
Mobile phase A: water/acetonitrile=90/10 v/v with 0.2% trifluoroacetic acid
Mobile phase B: water/acetonitrile=30/70 v/v with 0.2% trifluoroacetic acid
Elution: according to the following composition

| Time | Mobile phase A | Mobile phase B |
|---|---|---|
| 0 | 100 | 0 |
| 35 | 0 | 100 |
| 35.10 | 100 | 0 |
| 42.00 | 100 | 0 |

Flow rate: 1 ml/min
   Determination of assay value (enzymatic turbidimetric method using Micrococcus lysodeikticus cells): according to the FIP method (Pharmaceutical Enzyme, Ed 1997, 84, 375) and J Pharm Pharmacol. 2001; 53 (4); 549-54.

### EXAMPLES

### Example 1

### Chromatographic Purification of HEW

Absorption stage. 30 litres of HEW were loaded at a feed rate of 3 1/hour into a dryer with a Nutsche filter containing 2.9 litres of weakly acidic macroporous cationic polyacrylic resin having a particle size ranging between 300 and 1600 μm and a capacity≥2.7 eq/l, pre-conditioned with an aqueous solution of sodium carbonate (15% w/w) to pH 8.0, washed with water and flushed with nitrogen, the eluate being collected by gravity. 1 bed volume of demineralised water was then loaded, maintaining the resin under stirring, and the resulting suspension was maintained under stirring for 20 min, after which the water was drained by gravity; said treatment was repeated once more under the same experimental conditions.

Elution stage. The aqueous solution of 3.5% NaCl at 30-35° C. (1.8 bed volumes) is eluted by gravity.

Precipitation of crude lysozyme base. An aqueous solution of 6% NaOH (w/v) was added to the preceding 2% aqueous solution of crude lysozyme base under stirring at 20-25° C. until a final pH value of 10.5±2 was reached. The resulting solution was then cooled to 4° C. in 12-18 hours, and maintained at said temperature under stirring for 6 hours. The resulting precipitate was recovered by suction.

Preparation of amorphous powder of purified lysozyme hydrochloride. The wet solid obtained in the preceding step was dispersed under stirring at 35° C. in demineralised water (0.66 1) for 1 hour, and a 6% w/v aqueous solution of hydrochloric acid was then added until a final pH value of 2.9±0.1 was reached. The resulting solution was then heated under stirring at 40° C. for 1 hour, then cooled, and a 2% w/v aqueous solution of sodium hydroxide was added to reach a final pH value ranging between 8.5 and 9.0. Charcoal (powder) (2.5 g) was added to said solution, and the resulting mixture was stirred at room temperature for 2 hours; the resulting suspension was then filtered by suction, and the filter was washed with water (16 ml). The filtrate and washing water were collected, and a 6% w/v aqueous solution of hydrochloric acid was added at a temperature of less than 32° C. until a final pH value of 3.6±0.2 was reached. The resulting solution was then ultrafiltered (cut-off 10 kdaltons) to obtain a 30% w/v lysozyme hydrochloride content and diafiltered (cut-off 10 kdaltons) to remove the inorganic salts present. The resulting aqueous solution was then heated at 74° C. for 1 hour or alternatively at 90° C. for 2-6 minutes, then cooled to 40° C. and treated with a spray-dryer (desolvation chamber temperature 180° C.) to provide pure amorphous lysozyme hydrochloride as an ivory powder (105 g; recovery efficiency 99%).

The resulting product has an assay value (turbidimetric method) of 98.6% and an HPLC purity of 99.7%.

Evaluation of in vitro Antiviral Activity of Lysozyme HCl Against SARS-CoV-2

### Determination of Non-Toxic Concentration of Lysozyme HCl

Cell toxicity was monitored by establishing the effect of lysozyme HCl against Vero cells (epithelial cells of monkey kidneys). The cells were seeded in 96-well plates at the concentration of 1×10⁴ cells/well. 24 hours after seeding, the cells were treated with serial dilutions of lysozyme HCl or chloroquine (as control), in a final volume of 200 in triplicate. After 72 hours' incubation at 37° C. with 5% CO₂, cell viability was measured by MTT assay (D'Alessandro, M. et al., Differential effects on angiogenesis of two antimalarial compounds, dihydroartemisinin and artemisone: Implications for embryotoxicity, Toxicology. 241 (2007) 66-74. Doi: 10.1016/j.tox.2007.08.084).

The data were calculated as percentage cell viability using the formula:

[(absorbance of sample−blank of cell-free sample)/mean absorbance of control of media]×100.

The 50% cytotoxic concentration (CC50) that causes the death of 50% of Vero cells compared with the untreated control cells was determined. Visible morphological changes were observed with optical microscopy.

The CC50 of lysozyme HCl for Vero cells was determined as 13.3 mg/ml.

### Determination of Non-Toxic Concentration of Ovotransferrin and Chloroquine

The cytotoxicity of ovotransferrin and chloroquine (CQ) was measured by MTT assay, similarly to the method used for lysozyme HCl. Ovotransferrin proved non-toxic at the maximum concentration used (10 mg/ml) (100% viability compared with control cells). The CC50 and CC10 of CQ proved to be 95.3±18 and 20.93±4.39 respectively.

Isolation of SARS-CoV-2 from Nasopharyngeal Swabs SARS-CoV-2 was isolated from 500 μl of nasopharyngeal swabs, added to Vero cells at 80% confluence; the inoculum was removed after 3 hours' incubation at 37° C. with 5% CO₂, and the cells were incubated at 37° C., 5% CO₂, for 72 hours, when the cytopathic effects were evident.

The numbers of viral copies in the cell supernatant were quantified by specific quantitative real-time RT-PCR (qRT-PCR) ((World Health Organization (WHO). Coronavirus disease (COVID-19) technical guidance: Laboratory testing for 2019-nCoV in humans. US CDC Real-time RT-PCR Panel for Detection 2019-Novel Coronavirus (28 January 2020). Available at: https://www.fda.gov/media/134922/download [last access 20 March 2020].

SARS-CoV-2 was precipitated with PEG according to the manufacturer's instructions, and the viral load was determined by Plaque Assay, using dilution factors ranging between 10 and 10^9.

The virus was used with a multiplicity of infection (MOI) of 0.05 in the following experiments.

### Cell Infection and Treatment of Compounds

Vero cells were seeded in 96-well plates at a density of 1×10⁴ cells/well and cultured for 24 hours at 37° C. with 5% CO₂, they were then infected with an MOI of 0.05 (1000 PFU/well) and incubated for 2 hours at 37° C. with 5% CO₂. The virus was then removed, and the cells were treated with medium containing lysozyme HCl or chloroquine (as control) at different concentrations, and incubated for 72 hours at 37° C. with 5% CO₂. An additional protocol was conducted, adding a pre-incubation step: the virus (MOI 0.05) was incubated for 1 hour at 37° C. in the presence of various concentrations of lysozyme HCl before addition to the cell monolayer.

### Evaluation of Antiviral Activity of Lysozyme HCl

The numbers of viral copies in the cell supernatant were quantified by specific quantitative real-time RT-PCR (qRT-PCR).

The results (Table 1) were expressed as percentage viral replication, taking account of the replication in the untreated infected Vero cells, amounting to 100%.

Moreover, to verify the virucidal activity of the compound, a plaque test was conducted after inoculation into the cells of the virus plus the compound, plated in a 6-well plate. Briefly, after inoculation, the cells were covered with 0.3% agarose, dissolved in the cell medium and incubated for 72 hours at 37° C. with 5% CO₂. After removal of the agarose, the cells were fixed with a 4% formaldehyde solution and then stained with methylene blue. The plaques in each well were counted, and the results were expressed as plaque-forming units (PFU)/mL and as the ratio between PFUs in the untreated control and the treated cells.

TABLE 1
Evaluation of antiviral activity of lysozyme HCl

| Lysozyme HCl | Percentage viral replication | |
|---|---|---|
| concentration (mg/ml) | Cells pre-treated with | Lysozyme added to |
| tested | lysozyme HCl | already infected cells |
| 0.00 | 100 | 100 |
| 0.06 | / | 98 |
| 0.17 | / | 100 |
| 0.19 | 100 | 97 |
| 0.37 | 96 | 87 |
| 0.50 | / | 54 |
| 0.75 | 33 | 37 |
| 1.00 | 5 | 7 |
| 1.50 | 0 | 0 |
| 3.00 | 0 | 0 |

Each experiment was conducted in duplicate or triplicate, and two independent experiments were conducted.

### Antiviral Activity of Lysozyme HCl and Ovotransferrin Against SARS-CoV-2

Ovotransferrin alone was tested at different concentrations, ranging from 10 mg/ml to 1.25 mg/ml, but no antiviral activity was detected in said interval. Table 2 summarises the antiviral activity of lysozyme HCl combined with three different concentrations of ovotransferrin, expressed as ACt and percentage viral replication (average of three experiments). In all conditions, ovotransferrin increased the antiviral activity of lysozyme HCl. Interestingly, the lowest concentration of ovotransferrin (1.25 mg/ml) exhibited the highest synergic effect. The synergic antiviral effect obtained is dose-dependent.

Table 3 shows the comparative percentage viral replication data between lysozyme HCl and lysozyme HCl combined with ovotransferrin.

TABLE 2
Antiviral activity of lysozyme HCl and ovotransferrin against SARS-
CoV-2 expressed as percentage ΔCt and percentage viral replication

| Lysozyme HCl | Ovotransferrin | | Percentage |
|---|---|---|---|
| concentration | concentration | | replication of |
| (mg/ml) | (mg/ml) | ΔCt | SARS-CoV-2 |
| 0.32 | 1.25 | −9.3 | 62 |
| | 2.5 | −6.9 | 75 |
| | 5 | −4.3 | 83 |
| 0.42 | 1.25 | −11.7 | 33 |
| | 2.5 | −12.0 | 54 |
| | 5 | −6.3 | 79 |
| 0.56 | 1.25 | −17.9 | 25 |
| | 2.5 | −17.2 | 27 |
| | 5 | −11.8 | 54 |
| 0.75 | 1.25 | −24.6 | 0 |
| | 2.5 | −17.9 | 25 |
| | 5 | −19.0 | 29 |

TABLE 3
Comparison between lysozyme HCl and lysozyme HCl combined
with ovotransferrin; data expressed as percentage viral replication

| | | Percentage | |
|---|---|---|---|
| | | replication of | Percentage |
| Lysozyme HCl | Ovotransferrin | SARS-CoV-2 | replication of |
| concentration | concentration | (lysozyme + | SARS-CoV-2 |
| (mg/ml) | (mg/ml) | ovotransferrin) | (lysozyme only) |
| 0.32 | 1.25 | 62 | 93 |
| | 2.5 | 75 | |
| | 5 | 83 | |
| 0.42 | 1.25 | 33 | 89 |
| | 2.5 | 54 | |
| | 5 | 79 | |
| 0.56 | 1.25 | 25 | 93 |
| | 2.5 | 27 | |
| | 5 | 54 | |
| 0.75 | 1.25 | 0 | 62 |
| | 2.5 | 25 | |
| | 5 | 29 | |

Virucidal Activity To confirm the virucidal activity of lysozyme HCl, an assay was conducted on plaque using heat-treated lysozyme HCl (at 99° C. for 40 minutes) or non-heat-treated lysozyme HCl. Table 4 shows the results obtained, expressed as mean PFU/ml (average of three experiments). The infectivity of SARS-CoV-2 was reduced by 57.2%, 58.9% and 69.6%, using 0.42 mg/ml, 0.56 mg/ml and 1 mg/ml respectively. The non-heat-treated lysozyme HCl did not reduce the infectivity of SARS-CoV-2 at any of the doses used (Table 4).

TABLE 4
Virucidal activity of lysozyme HCl and heat-treated lysozyme HCl

| | Untreated | mean PFU/ml (average of three experiments) | | |
|---|---|---|---|---|
| | infected cells | 0.42 mg/ml | 0.56 mg/ml | 1 mg/ml |
| Lysozyme | 73.33 (95.5%) | 70 (83.9%) | 61.65 (84.1%) | 75 |
| HCl | | | | (<100%) |
| Heat-treated | 102.5 (100%) | 58.61 (57.2%) | 60.41 (58.9%) | 71.32 |
| lysozyme | | | | (69.6%) |

In Table 5, the enzyme activity and HPLC purity of samples of lysozyme HC1 heat-treated under different experimental conditions were comparatively analysed, evaluating different temperatures, dry treatment times (on the solid product) or treatment times in aqueous solution, by comparison with the activity expressed as degree of viral replication on an average of 9 experiments per sample. The data obtained confirm that all the heat treatments conducted give rise to high antiviral activity, and confirm the absence of thermal degradation (denaturation) in all the samples examined, except for the samples treated in aqueous solution for times exceeding 6 minutes, wherein a marked reduction in HPLC purity (−3 to −4%) and enzyme activity (−8 to −12%) was observed.

The absence of thermal degradation in the samples examined, confirmed initially by HPLC analysis and enzyme activity, was further confirmed by HSQC NMR (heteronuclear single quantum correlation nuclear magnetic resonance) analysis of the samples in D20 solution. The ¹H—¹³C—HSQC spectra of a sample of native lysozyme (not heat-treated) and a sample of lysozyme subjected to dry heat treatment (99° C. for 40 min) proved identical, confirming that the heat-treated lysozyme was not denatured.

TABLE 5
Lysozyme HCl heat-treated under different experimental conditions
vs. percentage viral replication and degree of denaturation

| | | % viral | | Enzyme |
|---|---|---|---|---|
| Types | Compound (test | replication | | activity |
| of heat | concentration in | (average of 9 | HPLC | (turbidimetric |
| treatment | mg/ml) | replications) | purity | method) |
| No heat | Lys HCl (0.75 mg/mL) | 25.51 | 99.7 | 98.7 |
| treatment | Lys HCl (0.5 mg/mL) | 58.13 | | |
| | Lys HCl (0.33 mg/mL) | 79.72 | | |
| Heat treat- | Lys HCl for 3 minutes | 15.55 | 99.4 | 97.3 |
| ment in | (0.75 mg/mL) | | | |
| aqueous | Lys HCl for 3 minutes | 67.97 | | |
| solution | (0.5 mg/mL) | | | |
| (90° C. | Lys HCl for 3 minutes | 87.85 | | |
| for the | (0.33 mg/mL) | | | |
| times | Lys HCl for 6 minutes | 9.88 | 99.0 | 97.4 |
| indicated) | (0.75 mg/mL) | | | |
| | Lys HCl for 6 minutes | 51.62 | | |
| | (0.5 mg/mL) | | | |
| | Lys HCl for 6 minutes | 87.96 | | |
| | (0.33 mg/mL) | | | |
| | Lys HCl for 20 minutes | 18.30 | 97.0 | 92.1 |
| | (0.75 mg/mL) | | | |
| | Lys HCl for 20 minutes | 36.10 | | |
| | (0.5 mg/mL) | | | |
| | Lys HCl for 20 minutes | 78.00 | | |
| | (0.33 mg/mL) | | | |
| | Lys HCl for 25 minutes | 2.62 | 96.4 | 88.8 |
| | (0.75 mg/mL) | | | |
| | Lys HCl for 25 minutes | 24.61 | | |
| | (0.5 mg/mL) | | | |
| | Lys HCl for 25 minutes | 42.06 | | |
| | (0.33 mg/mL) | | | |
| Dry heat | Lys HCl (0.75 mg/mL) | 16.58 | 98.8 | 98.1 |
| treatment | Lys HCl (0.5 mg/mL) | 35.56 | | |
| (99° C. | Lys HCl (0.33 mg/mL) | 60.32 | | |
| for 40 min) | | | | |
| Dry heat | Lys HCl (0.75 mg/mL) | 11.31 | 98.9 | 98.8 |
| treatment | Lys HCl (0.5 mg/mL) | 42.05 | | |
| (74° C. | Lys HCl (0.33 mg/mL) | 83.24 | | |
| for 60 min) | | | | |

1. Process for the purification of lysozyme HCl from hen egg white which comprises:
a) isolating crude lysozyme base from undiluted hen egg white using a weakly acidic cationic resin under stirring, followed by eluting with an aqueous saline solution;
b) preparing a crude solution of lysozyme HCl by treating the aqueous saline solution eluted in step a) with an aqueous inorganic base until a final pH value ranging between 10 and 11 is reached, at a temperature ranging between 0 and 8° for 4-24 hours, to obtain the lysozyme base which is first recovered by filtration, then dissolved in an aqueous solution of hydrochloric acid until a final pH interval of 2.5-3.5 is reached;
c) removing inorganic salts;
d) viral inactivating/antiviral activating;
e) isolating amorphous lysozyme HCl with a spray-drying technique;
f) heat treating the lysozyme HCl obtained in step e).

2. Process according to claim 1, wherein in step a), a weakly acidic polyacrylic macroporous cationic resin with a particle size range of 300-1600 μm, preconditioned to pH 7.0-9.0, is used.

3. Process according to claim 2, wherein in step a), the pH is adjusted with a 15% w/w aqueous solution of sodium carbonate.

4. Process according to claim 1, wherein in step a), the relative ratio between undiluted hen egg white and resin ranges between 8-12 l/l.

5. Process according to claim 1, wherein in step a), the resin is maintained under stirring at a stirring speed of up to 60 rpm and a temperature ranging between 25° C. and 40° C.

6. Process according to claim 1, wherein in step a), the weakly acidic cationic resin is Purolite® C106EP or equivalent, having a total capacity ≥2.7 eq/l.

7. Process according to claim 1, wherein in step a), the resin is eluted with a 2 to 7% NaCl solution at a temperature ranging between 25 and 40° C.

8. (canceled)

9. Process according to claim 1, wherein the aqueous inorganic base in step b) is a 4-8% w/v aqueous solution of sodium hydroxide.

10. Process according to claim 1, wherein in step b), the crude lysozyme base is dissolved in demineralised water at a relative ratio ranging between 1/30 and 1/60v/v relative to the initial amount of hen egg white.

11. Process according to claim 1, wherein in step c), the crude lysozyme hydrochloride solution is corrected to a final pH interval of 3.0-4.0, using a 2-8% aqueous solution of hydrochloric acid, and ultrafiltered and/or diafiltered to remove the inorganic salts.

12. Process according to claim 1, wherein step c) is conducted with ultrafiltration and diafiltration membranes with a cut-off of 10 kdaltons.

13. Process according to claim 1, wherein in step d), the ultrafiltered/diafiltered aqueous solution is heated to a temperature ranging between 40° C. and 100° C. for a period of up to 7 days.

14. Process according to claim 1, wherein in step f), the heat treatment of lysozyme hydrochloride is performed on the powder obtained in step e), at a temperature ranging between 40° C. and 100° C. for a period of up to 7 days.

15. Process according to claim 13, wherein the heat-treated lysozyme exhibits unchanged chromatographic purity and enzyme activity at the end of the treatment (±1.0%).

16. Process according to claim 1, wherein in step e), the solution obtained in step c) or d) is heated at 40° C. and treated with a spray-dryer.

17. Process according to claim 14, wherein, in step e), the temperature of the desolvation chamber ranges from 160 to 220° C. to provide pure amorphous lysozyme hydrochloride.

18. A method of protecting patients from SARS-CoV-2 infection, said method comprising
treating said patients with the lysozyme hydrochloride obtained by the process according to claim 1.

19. The method according to claim 18, wherein said lysozyme hydrochloride is administered in combination with antivirals and/or immunosuppressants selected from chloroquine, favilavir, remdesivir, avigan, tocilizumab, cyclosporin A, sirolimus, everolimus, temsirolimus, mycophenolate mofetil and pimecrolimus.

20. The method according to claim 19, wherein said lysozyme hydrochloride is administered in combination with ovotransferrin.

21. A method of preventing or treating SARS-CoV-2 in patients in need thereof, said method comprising
administering to said patients a pharmaceutical composition for oral, topical, inhalation, injectable, intravenous, gastrointestinal, intraperitoneal, intrapleural, intrabronchial, nasal or rectal administration, said pharmaceutical composition, comprising an effective antiviral amount of lysozyme hydrochloride, optionally combined with ovotransferrin with suitable carriers and/or excipients.
